(19) United States
(12) Patent Application Publication
(10) Pub. No.: US 2015/0080748 A1
(43) Pub. Date: Mar. 19, 2015

(54) Method and System for Predicting Cardiovascular Events

(71) Applicant: Laila Hübbert, Linkoping (SE)

(72) Inventor: Laila Hübbert, Linkoping (SE)

(21) Appl. No.: 14/391,765

(22) PCT Filed: Apr. 12, 2013
(86) PCT No.: PCT/IB2013/052935

§ 371 Date: Oct. 10, 2014

Related U.S. Application Data
(60) Provisional application No. 61/623,684, filed on Apr. 13, 2012.

(51) Int. Cl. A61B 7/00; A61B 5/00; A61B 5/02

(52) U.S. Cl. CPC A61B 7/005; A61B 5/02028; A61B 5/7275; A61B 5/746
USPC 600/481

(57) ABSTRACT

A method for predicting an increased risk for a complication in a patient subjected to mechanical circulatory support. The method includes continuously, or within given intervals, registering a acoustic intensity verses frequency curve from the mechanical circulatory support. Repeated sound intensity verses frequency curves are registered from a patient to obtain a mean curve for the patient. New sound intensity-frequency curves are repeatedly obtained and compared with the mean curve. Significant deviations between a new sound intensity-frequency curve and the mean curve are detected and indicate an increased risk for a complication event.

### CROSS REFERENCE TO RELATED APPLICATIONS

This application claims priority to U.S. Provisional Patent Application No. 61/623,684, filed on Apr. 13, 2012, entitled “Method for Prediction of Cardiovascular Events.” The entire disclosure of the foregoing provisional patent application is incorporated by reference herein.

### TECHNICAL FIELD

The present invention pertains to the prediction of the risk for adverse complications in connection with the use of mechanical circulatory supports in patients with heart failure. In particular, the invention pertains to the registration and analysis of sound frequency patterns, as an early stage indicator for events with an increased risk for the patient, in particular cardiovascular events.

### BACKGROUND

The use of mechanical circulatory support (MCS) has become an important treatment option for patients with advanced heart failure, and can be used in severe cases as a bridge to heart transplantation (BTT); or as a long-term palliative device, in destination therapy (DT), as an alternative to heart transplantation. Worldwide, over 13,000 patients with heart failure have been treated with the HeartMate II (HMII) (Thoratec Corporation, USA). The longest treatment period to date is more than seven years. One-year survival among patients supported with an MCS more than 30 days prior to heart transplantation is high, and the new continuous flow device has a survival rate at 1 year close to heart transplant patients. Patients who receive an MCS pending a heart transplant have lower creatinine and total bilirubin levels after two to four weeks of mechanical support, indicating improved organ perfusion and restoration of normal cardiac output. Further, MCS implantation improved diabetic control in patients with advanced heart failure. The MCS technology is continually improving, which results in a decrease in adverse events such as infection, septicemia and right heart failure, with shorter hospital stays and a favorable impact on both patient quality of life and treatment costs.

However, adverse thromboembolic events are still frequent, requiring the use of long-term anticoagulation with both anti-platelet drugs and warfarin. A higher prevalence of bleeding complications is associated with the use of HMII compared to the older pulsative devices such as the HeartMate XVE (from the same supplier). This higher prevalence of bleeding is not explained by excessive anticoagulation therapy alone, as previous work has shown that acquired von Willebrand syndrome occurs almost uniformly in patients on continuous-flow MCS, who develop bleeding complications, and this appears to contribute significantly to the condition. In addition to the physiological complications discussed above, malfunction of the MCS, for technical reasons, could result in very serious situations.

Acoustic signals from an assist device have been registered at various time intervals and analyzed, using a hydrophone data acquisition system with sensor, an AD converter and a data storage system correlated to the ECT. It was found that by acoustic signal monitoring it was possible to successfully identify HM SVE device end-of-life. Data from animal studies on an automatic diagnosis system designed for detection of early stage artificial heart malfunction of a pulsative device (undulation pump ventricular assist device) was published by Makino et al (Artif Organs 2006 30(5) 360-4). Their automatic diagnosis system was based on an electro-stethoscope system. An adaptive noise canceller was used to effectively eliminate ambient noise from the sound signal from the device detected by the electro-stethoscope, and a filtered sound signal was separated into frequency components by fast Fourier transformation. Frequency components of the pulsatile pump's acoustic signal were fed into the artificial neural network in order to diagnose the pump condition. By using this system it was possible to identify early signs of malfunction of the pump. However, these studies have focused on the pump as such and the function thereof.

### SUMMARY OF THE INVENTION

In one embodiment, the present invention is directed to a method of predicting an increased risk for a complication event in a patient subject to mechanical circulation support. The method includes recording acoustic intensity as a function of frequency continuously, or in a given frequency interval, for the mechanical circulation support of the patient. A mean acoustic intensity verses frequency curve for the patient is determined based on multiple acoustic intensity verses frequency recordings. Each subsequent additional acoustic intensity verses frequency recording is compared with the mean acoustic intensity verses frequency curve to detect a change between the additional recording and the mean intensity verses frequency curve. Significant changes between one or more new recordings and the mean curve indicates an increased risk for a complication event, such as a thromboembolic event.

In another embodiment, the invention is also directed to systems for predicting an increased risk for a complication event in a patient subject to mechanical circulation support. The systems comprise a. two or more microphones or accelerometers positioned so that the sound intensity frequency curve of the support is registered, b. a recording system where a series of sound intensity-frequency curves registered by the microphones or accelerometers are stored, c. an analyzing unit where one or more curves are compared with a mean curve based on earlier successive measurements during a given time interval, and d. an alarm system where a significant deviation between a mean curve and one or more successive curves in at least a section of the frequency interval initiates a signal to be sent to a supervising system.

Additional embodiments, features and advantages of the methods and systems according to the invention will be more fully apparent from the Detailed Description.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings.

FIG. 1 depicts a Box and Whisker plot of a stable sound intensity-frequency curve;

FIG. 2 illustrates a sound intensity-frequency curve for the initial week of acoustic recordings for a patient;

FIG. 3 illustrates a second sound intensity-frequency curve for the same patient as FIG. 2, depicting a significance change in the curve;

FIG. 4 illustrates a third, subsequent sound intensity-frequency curve for the same patient as FIG. 2;

FIG. 5 illustrates a fourth, subsequent sound-intensity-frequency curve for the same patient as FIG. 2, depicting a return to normal following an event;

FIG. 6 illustrates frequency analysis curves from an experimental setting;

FIG. 7 illustrates frequency analysis curves from a patient with an embolic stroke;

FIG. 8 is a plot of the variation in acoustic fingerprints at different pump speeds obtained experimentally using an HMII; and

FIG. 9 is a plot illustrating the experimental acoustic changes obtained from narrowing the inflow and outflow tubes, as well as from artificial thrombus and human thrombus.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention and, together with the description, serve to explain the principles of the invention; it being understood, however, that this invention is not limited to the precise arrangements shown.

### DETAILED DESCRIPTION

The following description of certain embodiments and examples should not be used to limit the scope of the present invention. Other features, aspects, and advantages of the versions disclosed herein will become apparent to those skilled in the art from the following description, which is by way of illustration, one of the best modes contemplated for carrying out the invention. As will be realized, the versions described herein are capable of other different and obvious aspects, all without departing from the invention. Accordingly, the drawings and descriptions should be regarded as illustrative in nature and not restrictive.

In the method described herein, acoustic patterns are monitored (e.g. sound intensity-frequency curves from a mechanical heart support device, in particular a continuous-flow device) to obtain indications of, not only the risk of malfunction of the device (i.e., a mechanical failure), but also patient related physiological status information. In particular, the method described herein provides a possibility for early detection of changes in the patient's physiological status, which can be used for early prediction of the risk for a certain disease or event, but even more importantly, of a situation which requires additional testing of various parameters for a complete diagnosis and/or prediction of the risk for complications. Conditions or complications of special interest are cardiovascular-related, like thromboembolic events, bleeding, infection, hypo- and hypervolemia, disposition of the in-and outflow of the pump, mechanical failure, etc. (i.e. situations which influence the interaction between the pump, heart and blood stream). Conditions or complications such as these produce changes in the acoustic pattern of the pump, e.g. within a given frequency interval, from the normal one for the patient. The major component of the pattern to be analyzed in accordance with this invention is the power of intensity, usually measured in decibels, over a given frequency interval (in Hz).

It has been discovered that the frequency analysis pattern curve is substantially constant over time for a given patient, as long as there are no complications of the type indicated above. However, in a situation with certain physiological changes, e.g. a cardio-vascular complication as thrombosis within the MCS, indicating the risk for a thromboembolic event, the frequency analysis pattern curve changes significantly, in particular at higher frequencies. Accordingly, a clear indication of a condition requiring attention from the doctor is provided. The additional parameters to be investigated after an event indicated by a pattern change includes proper physical examination, laboratory testing of various components of blood, blood pressure, electrocardiography (ECG), CT-scan, chest x-ray and ultrasonography ultrasonic.

Equipment for use in the present method includes one or more sensors which are designed to provide sound strength as a function of the frequency in a given frequency interval. Suitable sensors, including “microphones” among others, are well known and easily available on the market. Accelerometers may also be employed. The sensor(s) are positioned for recording the sound intensity-frequency and can be applied at the chest of the patient, or be positioned on or within the MCS. The signal covering a given time interval, e.g a few seconds up to several minutes or even longer, can be transferred to a recorder via a wire or sent wireless. Methods and tools for transferring, storing and monitoring acoustic data of this type are well-known, and therefore a detailed description of this type of component is not provided. The intensity (dB)—frequency (Hz) curves recorded at given time intervals are stored for the patient, and every new recording is compared with a mean curve based on the earlier curves. If a new curve differs significantly from the mean curve for the patient in a given frequency interval, this difference provides an indication of a condition requiring action from the medical staff. The significant difference in curves may be set up to trigger an alarm system for attention from the medical staff.

According to one aspect of the invention, a method is provided wherein acoustic intensity-frequency data are recorded over a given frequency interval and compared to the mean value recorded for the same patient. Significant deviations from the normal or mean curve over the whole, or sections of, the frequency interval is used for diagnosis of complications, in particular cardiovascular, such as thromboembolic events, bleeding, or infection.

According to one additional aspect of the invention, data from a series of patients equipped with an MCS are stored in a database. The database can comprise not only mean intensity-frequency curves from many patients, but also information about events leading to deviations from the normal or mean curves. The information about these events, and the analyses thereof, is also stored. Through recording information in a database from a great number of patients, and several events and their character, additional conclusions can be drawn at the event stage regarding possible events, or even the most likely event that has occurred. Sound intensity—frequency curves from a system carried by a patient can easily be transferred via Bluetooth or wi-fi to a central in the same room, a solution that preferably can be used in a hospital, or by a designed APP for a mobile telephone, for fully flexible use, where information from several patients can be stored and watched. A suitable frequency interval for measurement is from 0-30 000 Hz, and can be reduced to 0-22 000 Hz. At present, the interval 4 000 Hz to 19 000 Hz has been found to exhibit the most pronounced changes.

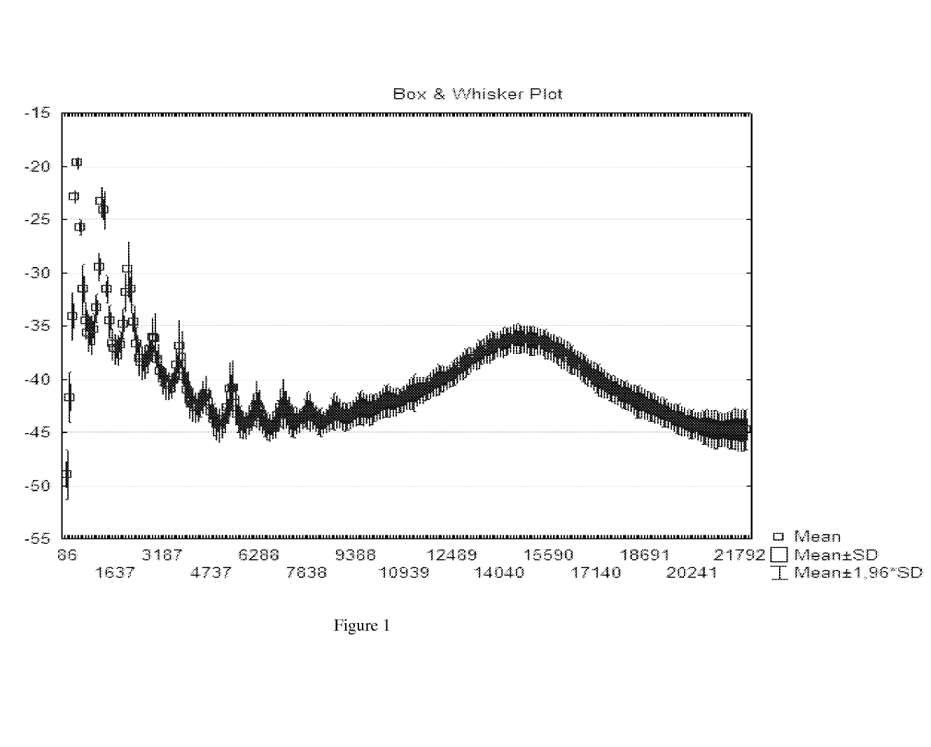

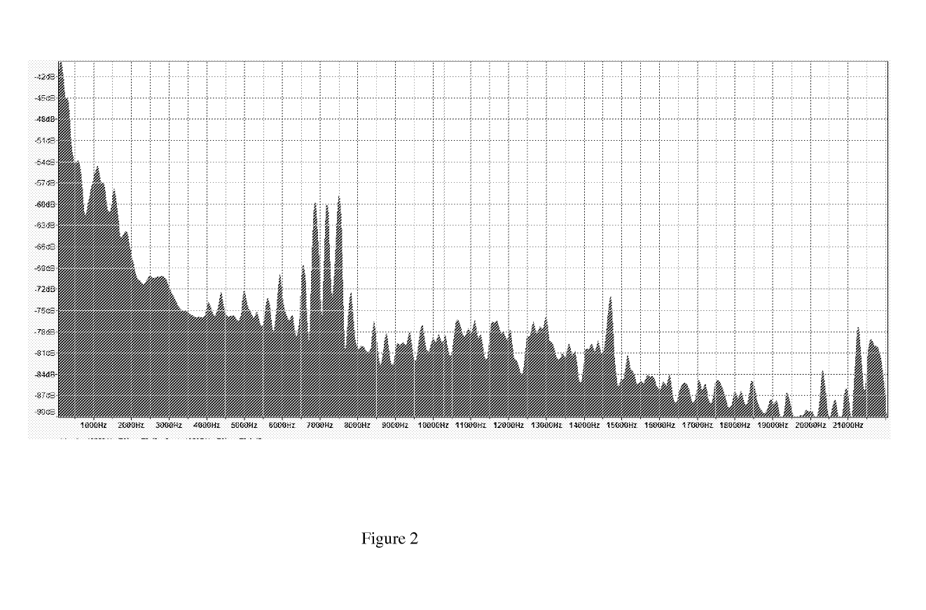

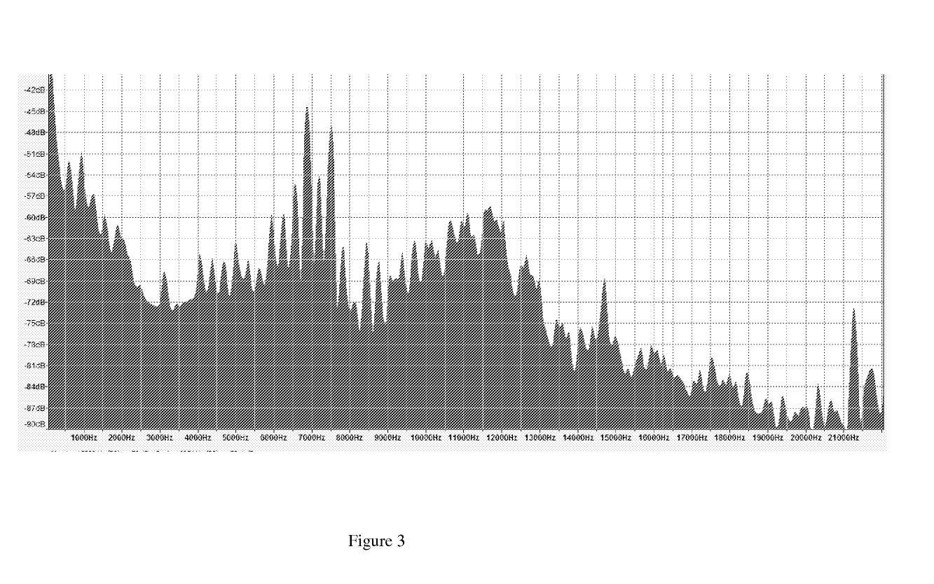

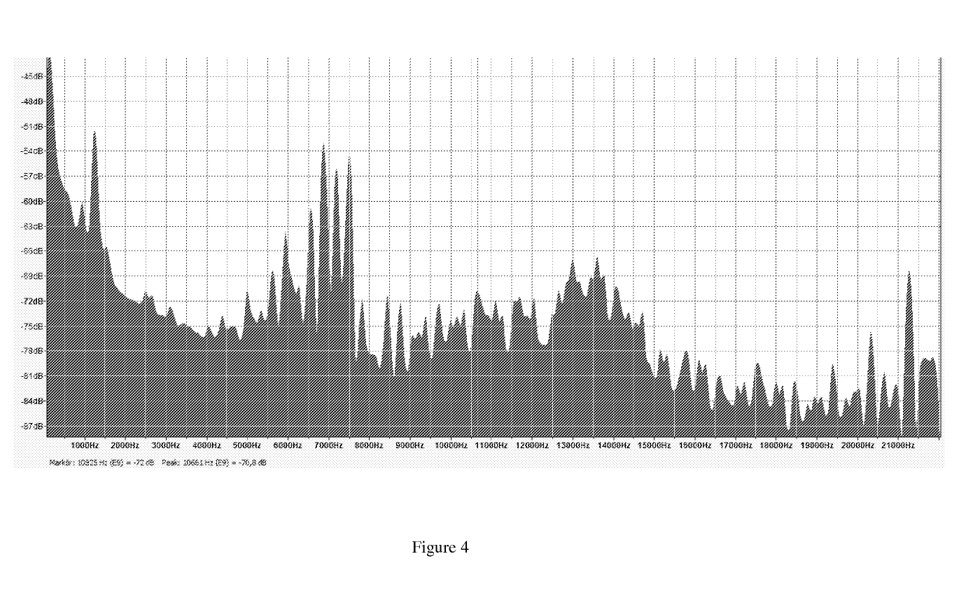

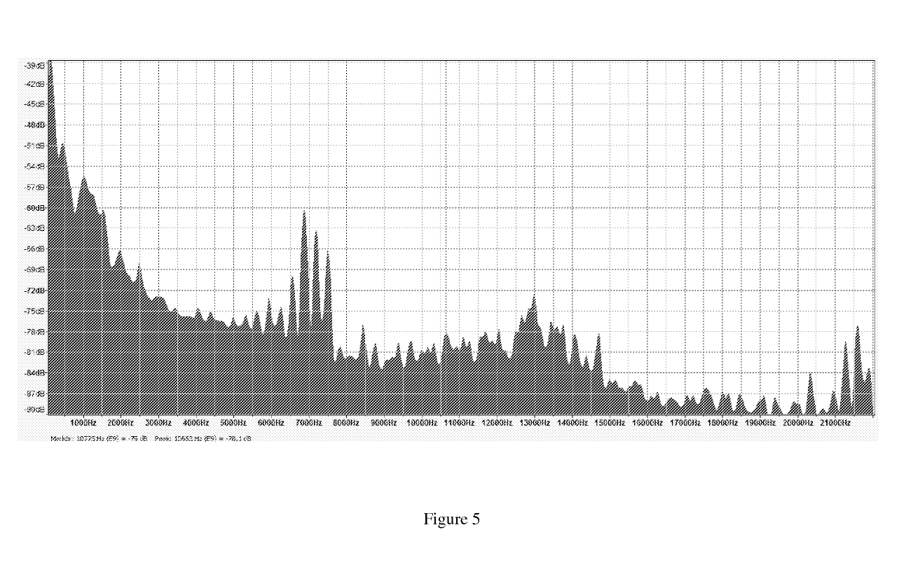

FIG. 1 depicts a frequency analysis curve from a clinically stable patient. The Box and Whisker plot of FIG. 1 shows a stable and reliable curve (x-axis: the frequency in Hz and y-axis: the amplitude in −dB). The method will be illustrated by the following example, using a sound intensity—frequency curve of a patient having an HMII device as a bridge to heart transplantation or as destination therapy. The curve from start and during the first week had the shape shown in FIG. 2. However, after a little more than one week, there was a significant change in the curve, in particular in the interval 9 000-19 000 Hz, for around 6 days, as shown in FIG. 3. The patient then suddenly had a stroke. It could be concluded that a few hours before the event happened the deviations from the original curve had started to decrease, as shown in FIG. 4. After relevant therapy and convalescence, the curve was the same as from start, as shown in FIG. 5.

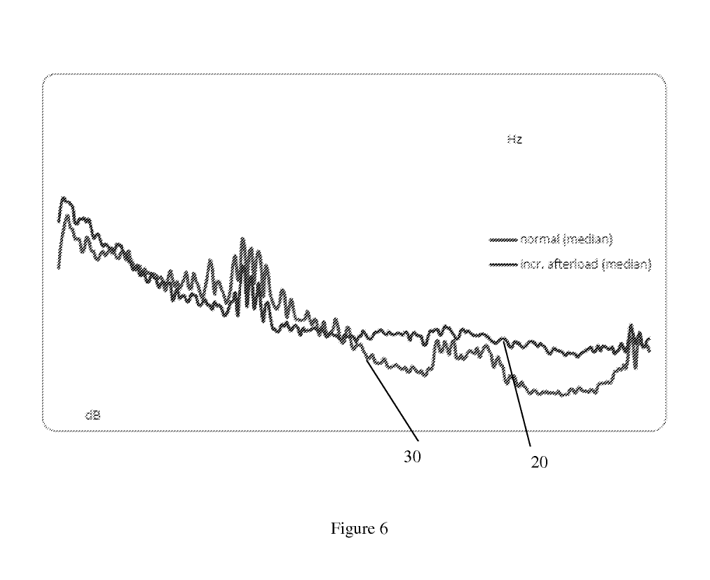

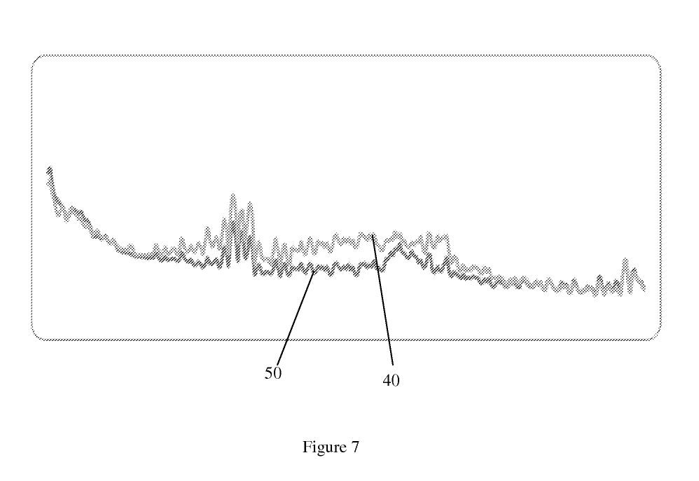

Frequency analysis curves from an experimental setting are illustrated in FIG. 6, where an increased afterload, which mimics the situation with thrombosis in the MCS system, is indicated by reference number 20. Frequency analysis curves at normal flow through the MCS, the lower curve at higher frequencies, is indicated by reference number 30. FIG. 7 depicts frequency analysis curves from a patient with an embolic stroke. Readings 3 days before the stroke are given by the upper curve. indicated by reference number 40, and can be compared to the mean curve for this patient (the lower curve) indicated by reference number 50. The deviations between the curves is most pronounced in the frequency interval of 4000 to 16 000 Hz.

These examples clearly illustrate the importance of the present method: by recording the sound intensity—frequency curve from a patient having a mechanical circulatory pump there is a good chance that a physiological complication event, in this case a stroke, is indicated before it actually happens. Sound intensity—frequency curves for three patients have been recorded with similar results, i.e. a physiological complication event can be foreseen with reasonable likelihood, approximately within days or a week before the event. Therefore, there should be a good chance for the doctor to carry out any complimentary test and diagnosis based on the indication from the system and initiate relevant prevention/therapy medication or surgery as deemed required.

### Experiment

A study of the efficacy of the method was performed using an experimental in-vitro model to register and analyze acoustic signals from a HMII continuous flow MCS. The aim of the study was to detect changes in sound correlating to artificial and human thrombosis, using modern telecom techniques.

The HMII was placed in a plastic bag filled with water to allow sound recordings via a smartphone and to mimic the clinical situation of sound recording. The smartphone's microphone was attached to the plastic bag at a distance of approximately 3 cm from the pump house. Baseline measurements were recorded with the HMII pumping at speeds of 6 000-10 000 rpm, and all the thrombus settings were performed at 8 000 rpm. Additional baselines were recorded with the ball valve connected to the out- and inflow tubing respectively. Thereafter, the pump out- and inflow tubing was narrowed sequentially (−50% of the diameter) to mimic thrombosis in the out- and inflow tubing. The ball valves were then removed and 20 ml of two different gelatin formulations with different viscosity (5 g and 10 g gelatin of animal origin in 2 dL water each) were injected into the inflow tubing. The pump was flushed between the experiments, which were repeated 3 times with each formula. A human thrombosis (20 ml) collected from a thoracic drainage in the Thoracic ICU, was finally injected into the inflow tubing.

### Acoustic Analysis

The sounds were recorded using an iPhone™ (Apple Inc. Cupertino, Calif., USA) with the commercially available stethoscope application iStethPro™ (Dr. Peter J Bentley) and transferred with modern telecom techniques to a frequency analysis software program Audacity™ 1.3.13-beta, (Unicode, Ash, Chinen and Crook) and analyzed at the different settings.

Sound is composed of many different waves with different frequencies (Hertz: fluctuations/unit time) and amplitudes (volts: sound strength, sound pressure, or noise level). In this analysis the description of amplitude is as noise level (−dB). It is possible to analyze the noise level at each frequency and also present the data in a graph with the amplitude at the y-axis in dB and the frequency at the x-axis in Hertz. The frequency measurements are set, by the commercially available sound analysis software program, between 0-23 000 Hz, and at 255 different standardized frequency levels. Six samples from each setting were collected and analysed. For each setting the HMII monitor power consumption (Watts), flow (L/min), and pump speed (rpm) were registered. All data are presented as mean, and changes in different parameters were calculated using the Wilcoxon's test for paired observations, and a p-level of <0.05 was regarded as significant

Results

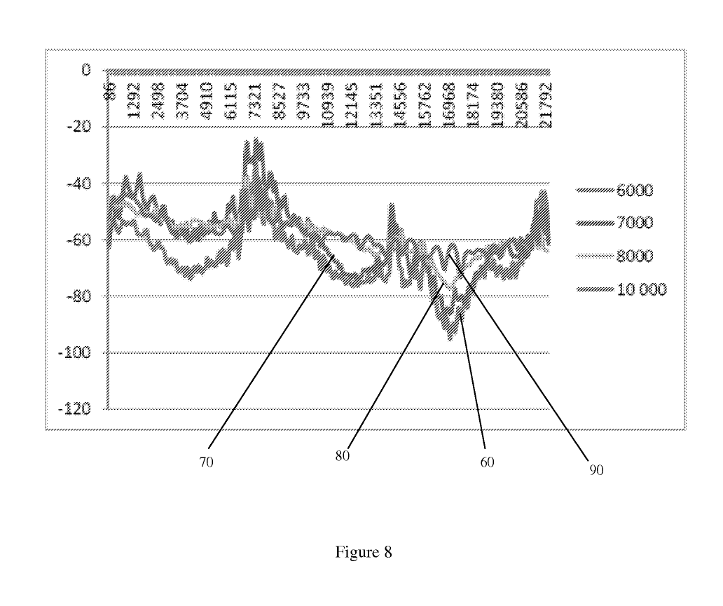

It was possible to collect and analyze the acoustic fingerprint from the HMII in the experimental setting using available telecom techniques. With this specific technique, the baseline acoustic fingerprint of this specific HMII was registered. Changes according to different pump speeds are shown in FIG. 8. The fingerprint corresponding to a pump speed of 6000 rpm is indicated by reference numeral 60, the fingerprint corresponding to a pump speed of 7000 rpm is indicated by reference numeral 70, the fingerprint corresponding to a pump speed of 8000 rpm is indicated by reference numeral 80, and the fingerprint corresponding to a pump speed of 10,000 is indicated by reference numeral 90. A significant (p<0.005) pan-spectrum change in sound strength was detected when the pump speed was increased. In FIG. 8, Frequency (x-axis) is in Hz, and amplitude (y-axis) is in −dB. All the baseline measurements at 8000 rpm between the different steps in the experiment settings showed the same baseline acoustic fingerprints, and the connected open ball-valves were not affecting the sound from the pump.

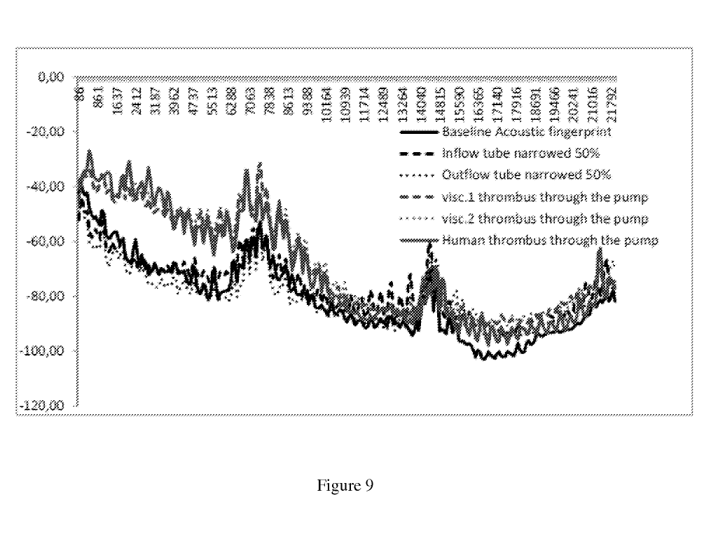

The frequency analysis pattern has the broadest frequency spectrum between 1 000 and 10 000 Hz. In this interval a major peak is seen at 1 000 Hz, and an additional smaller peak at ˜7 000 Hz may be present. At higher frequencies, peaks around ˜15 000 Hz and ˜22 000 Hz are commonly present. When the ball valves connected to the out- and inflow tubing were narrowed sequentially (˜50% of the diameter) to mimic thrombosis in the out- and inflow tubing, the change in the acoustic fingerprint was significant (p<0.005) in the high frequency spectrum, between 15 000-23 000 Hz. Similar acoustic changes were detected when artificial thrombosis was injected, or when a human thrombosis passed through the pump system. But at the experimental pump thrombus situation, the most significant changes (p<0.005) of the acoustic fingerprint were detected in the lower frequency spectrum between 0-10 000 Hz, as shown in FIG. 9 where frequency (x-axis) is in Hz, and amplitude (y-axis) is in −dB.

Acoustic changes from baseline at narrowing of the in- and outflow tubes were seen in high frequencies. Acoustic changes from artificial thrombus and human thrombus, passing through the pump system were seen both in low and high frequencies simultaneously. A significant (p<0.005) change in frequencies from the baseline acoustic fingerprint was detected at all experimental settings. After narrowing the in-and outflow tubing, the power decreased and the flow was reduced to 43% of the original output. When the artificial thrombus passed through the pump, there was a decrease in power. but when the human thrombus passed through the pump, there was a significant increase of power shown on the HMII monitor. There was no flow data presented on the monitor when the 3 different thromboses passed through the pump. Table 1 lists the data from the pump monitor at the different settings.

TABLE 1
Data shown on the pump monitor during the different settings.

| | Power | Flow |
|---|---|---|
| | (W) | (L/min) |
| Baseline Acoustic fingerprint | 4.2 | 3.9 |
| Inflow tube narrowed 50% | 3.2 | 1.7 |
| Outflow tube narrowed 50% | 3.2 | 1.7 |
| visc. 1 thrombus through the pump | 2.7 | na |
| visc. 2 thrombus through the pump | 2.4 | na |
| Human thrombus through the pump | 7.4 | na |

In this study, it was possible to register sound from the HMII device and to define an acoustic fingerprint using available telecom techniques in the experimental setting. In this experimental model, artificial thrombosis as well as human thrombosis were employed in an attempt to mimic the clinical situation of a thromboembolic event. With this specific technique and baseline acoustic spectrum, a baseline acoustic fingerprint, unique for this HMII was identified. The acoustic fingerprint, and the changes in frequency patterns, were similar to those previously observed in patients with and without complications. As seen in results from our ongoing clinical study, where the acoustic fingerprint of patients varied with pump speed, this study shows that a change in pump speed induces a similar change in the baseline sound spectrum. The current study has not shown if a change in the acoustic fingerprint signifies turbulence, a vortex phenomenon, large eddies from restriction of the in- and/or outflow, or a normal change in the pumps natural frequency range. Since the peaks at ˜7 000, ˜15 000, and ˜22 000 Hz are commonly present, they may be second harmonics due to the characteristics of sound waves in liquid and/or the size and configuration of the pumps and may have no clinical significance. Since the HMII has its own acoustic fingerprint in the experimental setting, as well as in the clinical situation, it is the change from a baseline acoustic fingerprint (at a given pump speed) that appears to be a change of importance and maybe not a specific amplitude or frequency patterns.

This study shows a similar change of higher frequency spectrum in a model with internal narrowing of the in- and outflow tubes as when an artificial thrombus and human thrombus passed through the pump system, and an additional specific lower frequency spectrum change when an artificial thrombus and human thrombus passed through the pump system. This change of lower frequency spectrum indicates that the change in acoustic fingerprints may detect where the thrombus is located in the pump system.

In the clinical setting, a sudden increase in power consumption at a fixed pump speed has aroused suspicion of pump thrombosis. The sensitivity of this phenomenon is not known due to diagnostic problems with current available diagnostic methods of detecting pump thrombosis, including Echocardiography, CT scanning and blood samples. It is well-known from clinical experience that neurological events including stroke may be the first symptom suffered by a patient despite the lack of change in power consumption of the pump. The results from the current study and early findings indicate that acoustic analysis may provide a new means of detecting pump thrombosis in patients treated with a continuous flow assist device.

While several methods and components thereof have been discussed in detail above, it should be understood that the components, features, configurations, and methods discussed are not limited to the contexts provided above. In particular, components, features, and configurations, described in the context of one of the methods may be incorporated into any other methods. Furthermore, not limited to the further description provided below, additional and alternative suitable components, features, configurations, and methods, as well as various ways in which the teachings herein may be combined and interchanged, will be apparent to those of ordinary skill in the art in view of the teachings herein. Having shown and described various versions in the present disclosure, further adaptations of the methods and systems described herein may be accomplished by appropriate modifications by one of ordinary skill in the art without departing from the scope of the present invention. Accordingly, the scope of the present invention should be considered in terms of the following claims and is understood not to be limited to the details of structure and operation shown and described in the specification and drawings.

I claim:

1. A method for prediction of an increased risk for a complication event in a patient subject to mechanical circulation support, the method comprising the steps of:
a. recording acoustic data from the mechanical circulation support of the patient;
b. representing the acoustic data as an intensity verses frequency curve;
c. determining a mean intensity verses frequency curve for the patient based on multiple acoustic data recordings; and
d. comparing each additional recording of acoustic data with the mean intensity verses frequency curve to detect a change between the additional acoustic data recording and the mean intensity verses frequency curve.

2. The method of claim 1, wherein the detected change occurs across a whole frequency interval.

3. The method of claim 1, wherein the detected change occurs in a section of a frequency interval.

4. The method of claim 1, wherein the acoustic data is recorded at multiple given time intervals.

5. The method of claim 1, wherein the acoustic data is recorded continuously.

6. The method of claim 1, wherein the intensity verses frequency curve for the patient is compared to a database of mean intensity verses frequency curves for multiple patients to analyze a risk of a complication event.

7. The method of claim 1, wherein an alarm is produced upon detection of a significant change between the intensity verses frequency curve of one or more acoustic readings and the mean intensity verses frequency curve for the patient.

8. The method of claim 1, wherein a significant change between one or more additional acoustic data recordings and the mean intensity verses frequency curve indicates an increased risk for a complication event.

9. The method of claim 8, wherein the complication event is a thromboembolic event.

10. The method of claim 8, wherein the complication event is a mechanical failure.

11. A method of predicting an increased risk of a complication event in a patient using a mechanical heart support device, the method comprising:
a. monitoring an acoustic pattern from the mechanical heart support device;
b. analyzing the monitored acoustic pattern by comparing the pattern to a mean based on earlier acoustic patterns for the patient; and
c. detecting significant changes in the acoustic pattern compared to the mean pattern, the detected changes being indicative of an increased risk of a complication in a physiological condition of the patient.

12. The method of claim 11, wherein the monitoring step includes registering sound intensity as a function of frequency in a given frequency interval.

13. The method of claim 12, wherein the detected changes indicative of an increased risk of a complication occur in higher frequencies of the acoustic pattern.

14. The method of claim 12, wherein the frequency interval is 4000 Hz to 19000 Hz.

15. The method of claim 12, wherein the frequency interval is up to 30000 Hz.

16. A system for prediction of an increased risk for a complication event in a patient subject to mechanical circulatory support, comprising:
a. two or more microphones or accelerometers positioned so that the sound intensity frequency curve of the support is registered;
b. a recording system where a series of sound intensity-frequency curves registered by the microphones or accelerometers are stored;
c. an analyzing unit where one or more curves are compared with a mean curve based on earlier successive measurements during a given time interval; and
d. an alarm system where a significant deviation between a mean curve and one or more successive curves in at least a section of the frequency interval initiates a signal to be sent to a supervising system.
